(19) United States
(12) Patent Application Publication
(10) Pub. No.: US 2004/0067240 A1
(43) Pub. Date: Apr. 8, 2004

(54) Therapeutic agent comprising a b-subunit of a protein toxin

(76) Inventors: Andrew John Morgan, Bristol (GB); Andrew Douglas Wilson, Bristol (GB); Kong Wee Ong, Bristol (GB)

Correspondence Address: BOZICEVIC, FIELD & FRANCIS LLP, 200 MIDDLEFIELD RD, SUITE 200, MENLO PARK, CA, 94025, US

(21) Appl. No.: 10/450,334

(22) PCT Filed: Dec. 11, 2001
(86) PCT No.: PCT/GB01/05452

(30) Foreign Application Priority Data
Dec. 11, 2000 (GB) 0030067.3

(51) Int. Cl. A61K 39/295; C07K 14/005

(52) U.S. Cl. 424/201.1; 530/350; 530/395

(57) ABSTRACT

A B-subunit of a protein toxin selected from the B-subunit of E. coli heat-labile enterotoxin (EtxB) and the B-subunit of Vibrio cholerae toxin (CtxB) has a therapeutic effect against cell surface-expressed viral antigens and tumour antigens. In particular, the protein toxin may be used to treat an animal body, including human, suffering from a disease or condition associated with Epstein Barr Virus or suffering from neoplasia. The therapeutic agent may, additionally, comprise a cell surface-expressed antigen, for instance an Epstein Barr Virus latent membrane protein.

This invention relates to a therapeutic agent. More particularly, the present invention relates to a therapeutic agent comprising a B-subunit of a protein toxin, which may be useful in the treatment of a viral infection, to a fusion protein comprising such a B-subunit, to the use of a composition comprising such a B-subunit in the manufacture of a medicament and to a method of treatment of an animal body.

About 90% of the world's population are carriers of Epstein-Barr virus (EBV) by early adulthood, making it one of the most common human viral infections.

Most primary EBV infections are asymptomatic or subclinical in presentation. In a minority of cases, it manifests clinically as infectious mononucleosis (glandular fever), which is a self-limiting, febrile illness characterised by a generalised rash, arthralgia, lymphadenopathy and hepatosplenomegaly.

Of perhaps greater significance is the association of EBV with a number of human tumours of epithelial and lymphoid origin. These include nasopharyngeal carcinoma, Burkitt's lymphoma, Hodgkin's lymphoma and post-transplant lymphoproliferative disease.

EBV is able to transform B-cells in-vitro, forming lymphoblastoid cell lines (LCLs). These LCLs closely resemble activated B-cells but, additionally, they express the whole complement of latent EBV genes, displaying a latency III pattern. These genes code for nuclear antigens EBNA1, 2, 3A, 3B, 3C and LP, latent membrane proteins LMP1 and 2 as well as two small RNAs known as EBER1 AND 2.

However, in infected B-cells in-vivo and most EBV related tumours, the range of latent genes expressed in some is limited to LMP2 (latency 0), EBNA1 (latency I), with the addition of LMPs and EBERs in others (latency II).

During primary infection, the host is able to generate strong humoral and cellular responses. However, infected cells displaying the latency 0, I and II patterns are able to evade immune surveillance and viral clearance is not achieved. This enables the virus to establish a life-long reservoir within the host B-cell pool. The transformation and immortalisation of host B-cell is believed to be a crucial step towards EBV carcinogenesis, although the actual mechanism remains unresolved.

It has become increasingly clear that cytotoxic T-cell (CTL) responses against EBV are of central importance in the control of established latent infection (1). Indeed, current research has concentrated on the generation and expansion of CTLs against the various latent gene products, which could provide the basis for immunotherapy against EBV related tumours.

A key feature of CTL responses against EBV is the immunodominance of EBNA3A, 3B and 3C, with weaker responses against LMP2, EBNA2 and rarely, LMP1 (2). EBNA1 is rendered non-immunogenic by the presence of an internal glycine-alanine repeat (GAr) domain (3). With most EBV-associated tumours exhibiting a latency I or II pattern, emphasis has been placed on enhancing anti-LMP1 or 2 CTL responses.

The B-subunit of E. coli heat-labile enterotoxin (EtxB), and its closely related homologue, CtxB, the B subunit of Vibrio cholerae toxin, attaches onto the surfaces of target cells via its receptor GM1, a ubiquitous cell-surface glycosphingolipid, and this results in rapid aggregation or capping of bound EtxB/CtxB, which is then followed by internalisation of the toxin.

EtxB has been shown to improve the uptake of exogenous antigens across mucosal surfaces as well as the immunogenicity of these antigens. Hence it is potentially useful as an adjuvant in the design of mucosally-delivered vaccines (4).

The EBV latent membrane proteins, LMP1 and 2, are found on LCLs and several EBV-associated malignancies including nasopharyngeal carcinoma (5) and Hodgkin's disease. LMP1 has been shown to be highly concentrated on the plasma membrane in glycosphingolipid-rich (GSL) domains (6). In a separate finding, LMP2 was shown to colocalise with LMP1 by fluorescence microscopy (7). GM1 is also found in abundance within these domains.

Without wishing to be bound by theory, the present inventors believe that following the addition of a non-toxic recombinant form of EtxB to LCLs, LMP1 and 2 might undergo aggregation and internalisation with EtxB as they are found in the same GSL domains as the EtxB receptor GM1. These viral proteins might then undergo an alternative antigen processing pathway, which could ultimately result in previously protected epitopes on these proteins being processed and presented in a more efficient manner to cytotoxic T-lymphocytes. It is believed that this theory extends to CtxB and, moreover, to cell surface-expressed viral antigens generally and not only to EBV latent membrane proteins.

Accordingly, the present invention provides, in a first aspect, the use of a B-subunit of a protein toxin selected from the B-subunit of E. coli heat-labile enterotoxin (EtxB) and the B-subunit of Vibrio cholerae toxin (CtxB) to alter antigenic processing and presentation of viral and tumour antigens. The viral antigens are cell-surface expressed viral antigens, especially EBV latent membrane proteins. Thus, in a second aspect, the present invention provides the use of a B-subunit of a protein toxin selected from the B-subunit of E. coli heat-labile enterotoxin (EtxB) and the B-subunit of Vibrio cholerae toxin (CtxB) in the manufacture of a medicament for the treatment of a disease or condition associated with the Epstein Barr Virus in an animal body, including a human body, infected with Epstein Barr Virus. According to a third aspect, the present invention provides the use of a B-subunit of a protein toxin selected from the B-subunit of E. coli heat-labile enterotoxin (EtxB) and the B-subunit of Vibrio cholerae toxin (CtxB) in the manufacture of a medicament for the treatment of neoplasia in an animal body, including a human body.

It will be clear that the B-subunit of a protein toxin selected from the B-subunit of E. coli heat-labile enterotoxin (EtxB) and the B-subunit of Vibrio cholerae toxin (CtxB) has an effect on diseases or conditions in an animal body, including a human body, in which viral cells or tumour cells bearing cell surface-expressed antigens are involved pathogenically. Thus, an animal body, including a human body, infected with, or carrying, Epstein Barr Virus can be treated therapeutically by the administration of an effective amount of a composition which comprises EtxB or CtxB. Furthermore, an animal body, including a human body, suffering from a neoplasia can be treated therapeutically by the administration of an effective amount of a composition comprising EtxB or CtxB.

The composition comprising EtxB or CtxB may additionally comprise a cell surface-expressed antigen.

Accordingly, the present invention provides in a further aspect a therapeutic agent comprising a B-subunit of a protein toxin selected from the B-subunit of E. coli heat-labile enterotoxin (EtxB) and the B-subunit of Vibrio cholerae toxin (CtxB) and a cell surface-expressed antigen. Preferably, the cell surface-expressed antigen is a cell surface-expressed viral antigen, particularly an Epstein Barr Virus latent membrane protein. This may be EBV LMP1 or EBV LMP2.

Conveniently, the B-subunit of the protein toxin and the cell surface-expressed viral antigen are linked, or are conjugated. Alternatively, the B-subunit of the protein toxin and the cell surface-expressed viral antigen may be fused.

The present invention further provides a fusion protein comprising the B-subunit of the protein toxin, preferably EtxB, and a cell surface-expressed viral antigen, which is preferably an EBV latent membrane protein.

Methods by which fusion proteins may be produced are, of course, well known in the art. It is, thus, believed that the fusion proteins disclosed herein may be produced according to known techniques and procedures. In this respect, reference is made to U.S. Pat. No. 5,589,384, EP-A-0418626 and WO 00114114.

The present invention additionally provides a fusion protein comprising a first protein homologous to the B-subunit of either E. coli heat-labile enterotoxin (EtxB) or Vibrio cholerae toxin (CtxB) and a second protein homologous to a cell surface-expressed viral antigen, said first homologous protein being capable of binding to the GM1-receptor and said second homologous protein being capable of being intemalised into a cell and altering the antigen processing pathway therein. The agent of the present invention may be used in the treatment of viral diseases. In the case where the cell surface-expressed viral antigen is an EBV latent membrane protein the agent can be used to treat diseases associated with EBV or in the treatment of neoplasia, for instance leukaemia. Hence, the present invention further provides a method of treating an animal body, including a human body, suffering from an Epstein Barr related illness which comprises administering to the animal body, including the human body, an effective amount of the therapeutic agent. The invention also provides a method of treating an animal body, including a human body, suffering from a neoplasia which comprises administering to the animal body, including the human body, an effective amount of the therapeutic agent.

The therapeutic agent of the present invention comprising the B-subunit of the protein toxin, or additionally comprising the cell surface-expressed antigen, may be administered to an animal body, including a human body, in the form of a pharmaceutical composition which comprises, in addition to the therapeutic agent, one or more pharmaceutically-acceptable carrier, diluent or excipient. The therapeutic agent, either itself or in the form of a pharmaceutical composition, may be administered for a variety of preventative and therapeutic purposes and administration may be by any of the means which are conventional for pharmaceutical agents, including oral and parenteral means.

The present invention will now be described, by way of example only, with reference to the accompanying drawings of which:—

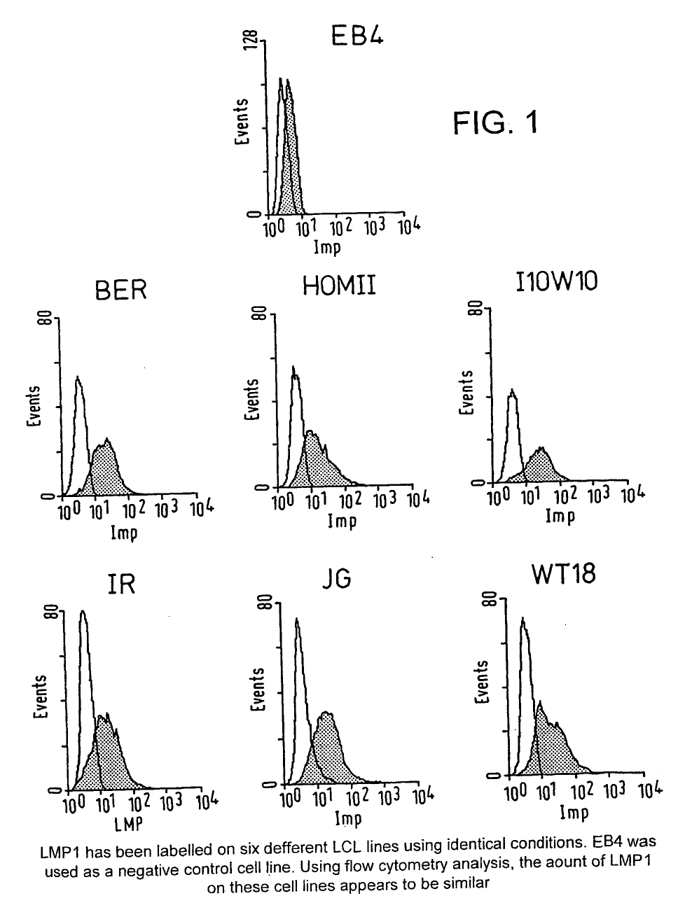

FIG. 1 is a series of graphs showing labelling of six different LCL lines with LMP1 using identical conditions;

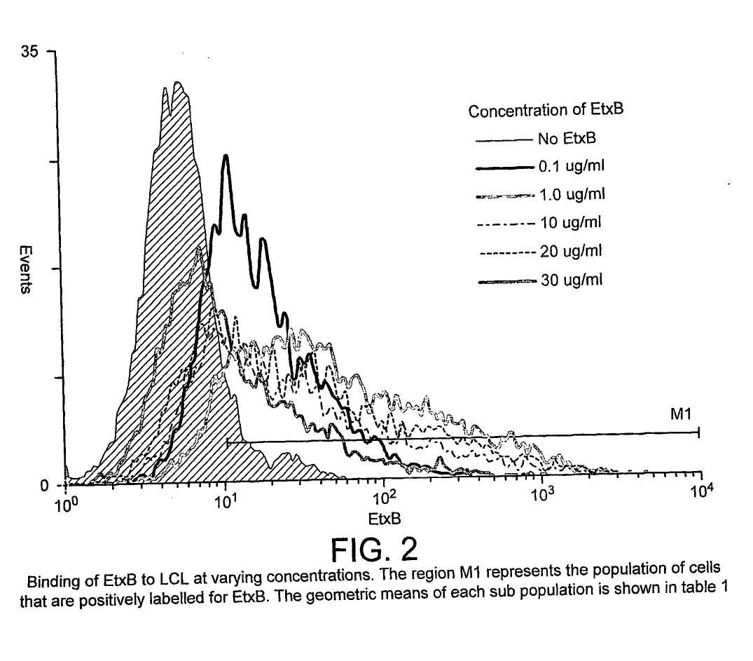

FIG. 2 is a graph showing binding of EtxB to LCL at varying concentrations;

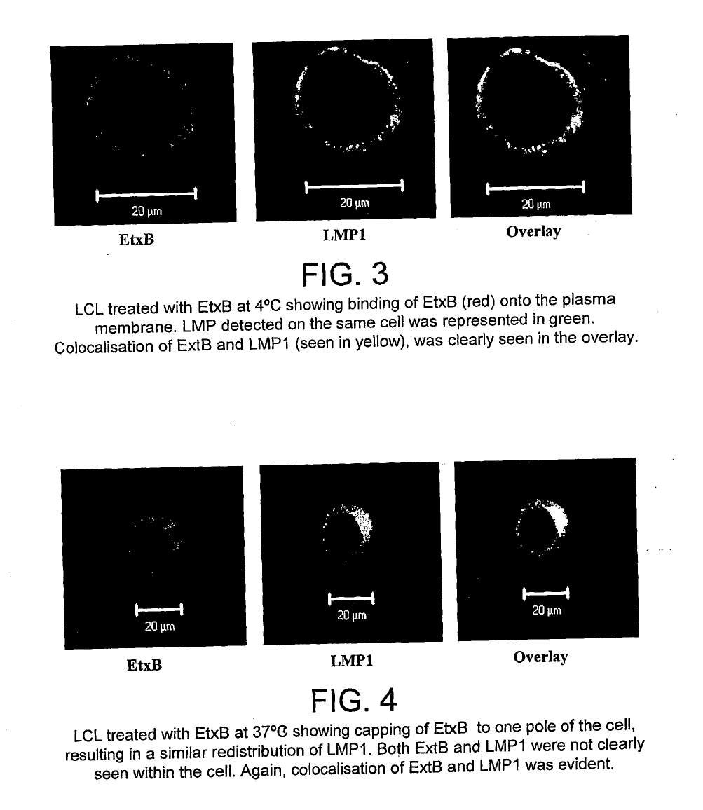

FIG. 3 is an immunofluorescence micrograph showing binding of EtxB onto the plasma membrane;

FIG. 4 is an immunofluorescence micrograph showing capping of EtxB to one pole of a cell;

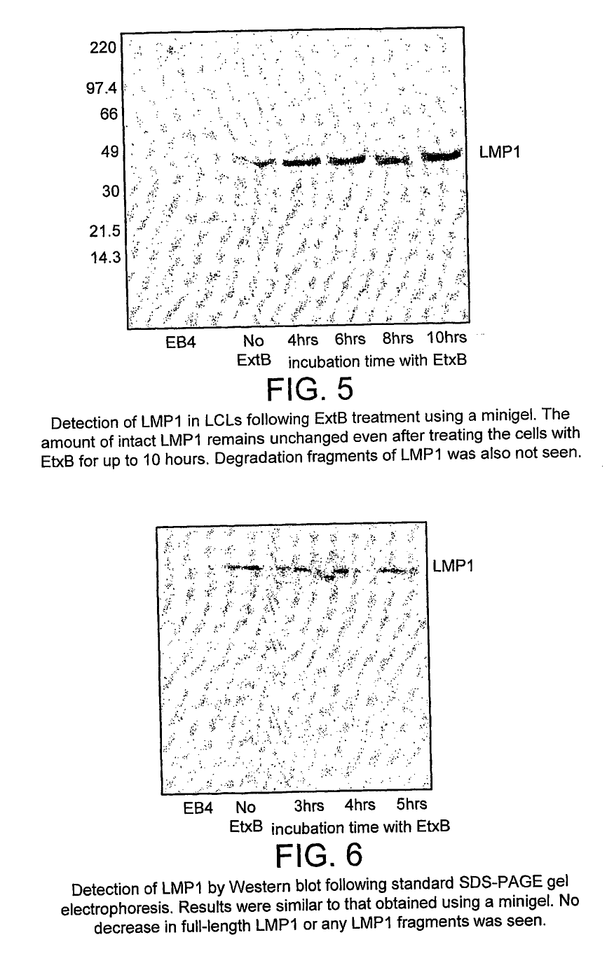

FIG. 5 is a western blot showing detection of LMP1 in LCLs following EtxB treatment;

FIG. 6 shows detection of LMP1 by western blot following SDS-PAGE;

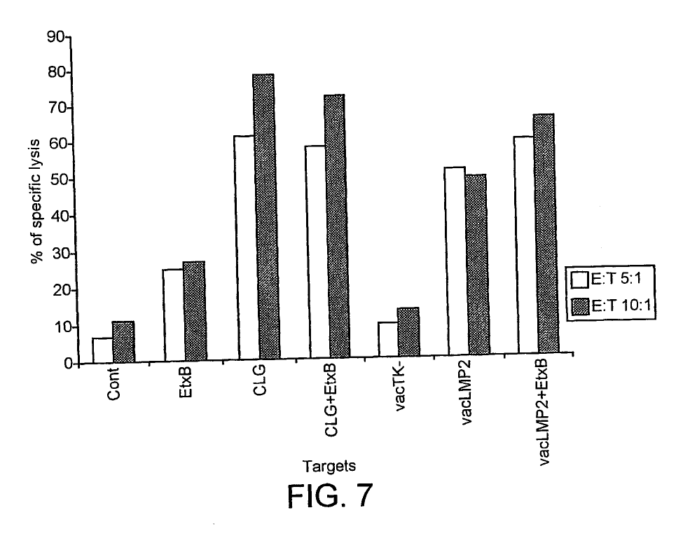

FIG. 7 is a bar chart comparing cytotoxic activity of WT poly T against different target cells, and

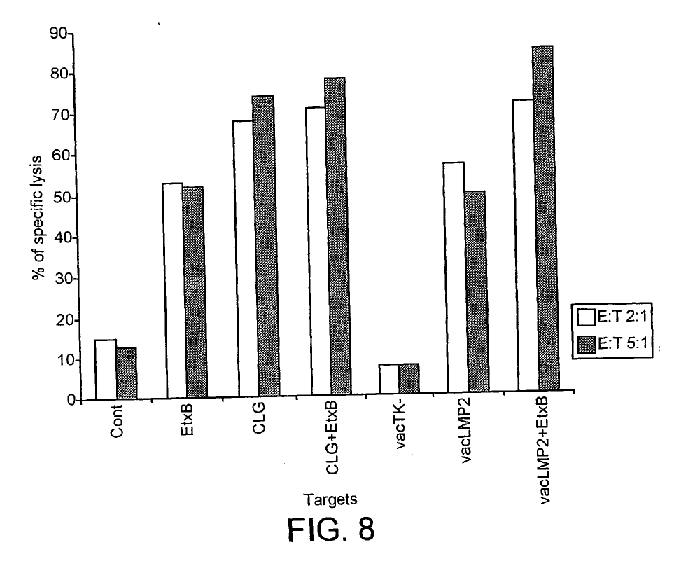

FIG. 8 is a graphical representation of CTL activity of DA c64 against different targets.

### EXPERIMENTAL

### A

### Cell Culture

Lymphoblastoid cell lines (LCLs) originated from various donors were used in the experiments. EB4 is a EBV-negative B-cell lymphoma cell line and used as negative control. These cells were cultured in RPMI1640 medium supplemented with 10% fetal calf serum, 2 mM glutamine, 100 μg/ml penicillin and 100 μg/ml streptomycin (complete RPMI medium) at 37° C. in a 5% CO₂ humidified atmosphere. Viability of cells were determined by Trypan blue dye exclusion.

### Metrizamide Treatment

In some experiments, debris was separated from healthy cells by underlying 1 ml of metrizamide (18% w/v metrizamide and 2% FCS in PBS) solution beneath 2 ml complete RPMI medium containing LCLs and spun at 500 g for 15 min. Viable cells were then carefully collected from the interface and washed.

### EtxB and Antibodies

Non-toxic recombinant EtxB, 118.8 monoclonal mouse anti-EtxB antibody and rabbit anti-EtxB serum were kindly provided by Prof. T. R. Hirst, University of Bristol. CS1-4 mouse anti-LMP1 antibody (Dako), FITC-conjugated goat anti-mouse IgG antibody (Sigma), Texas red-conjugated donkey anti-rabbit IgG antibody and FITC-conjugated donkey anti-mouse IgG antibody (both from Jackson lmmunoResearch).

### Treating Cells with EtxB

LCL cultures containing 1×10⁶cells/ml were incubated with 10 μg/ml of EtxB were placed in 2 ml wells at 37° C. in a 5% CO₂ humidified atmosphere for the times specified in each experiment. These conditions are compatible with the induction of capping and internalisation. This process was stopped by washing in ice-cold PBS containing 0.1% azide. Negative control cells were incubated at the above conditions but in the absence of EtxB. A second set of negative control consisted of cells treated with EtxB but incubated at 4° C. which allow surface binding but inhibit capping and internalisation.

### Flow Cytometry

For detection of LMP1 and LCLs, cells were fixed using 1% paraformaldehyde in phosphate lysine buffer for 1 hour at 4° C. They were then indirectly labelled for LMP1 using CS1-4 (1:100) followed by FITC conjugated anti-mouse IgG (1:100). For determining the binding affinity of EtxB onto LCLs, the cells were treated with varying concentrations of EtxB for 1 hour at 4° C., washed and then labelled for EtxB using 118.8 (1:20) as primary antibody and FITC-conjugated anti-mouse IgG antibody (1:100) as secondary antibody. For both experiments, cells were washed once after they were appropriately stained and analysed using a Becton Dickinson FACScan system.

### Confocal Microscopy

Cells were treated with EtxB as stated above for 4 hours at 4° C. or 37° C. before fixation using 1% paraformaldehyde in phosphate lysine buffer was done. These were first treated with CS1-4 (1:50) and rabbit anti-EtxB serum (1:500) followed by FITC-conjugated donkey anti-mouse IgG antibody (1:100) and Texas red-conjugated anti-rabbit IgG antibody (1:500) as second-stage antibodies. Cell pellets were then resuspended in 10-20 ul of Vectashield mounting medium (Vector) and mounted onto slides. Simultaneous visualisation of LMP1 and EtxB was performed at 40× and 60× magnification using a Zeiss upright scanning confocal microscope.

### Gel Electophoresis and Western Blotting

Pre-cast Bis-Tris minigel set (Novex) as well as standard SDS-polyacrylamide gels were used for gel electophoresis and western blotting to detect LMP1 in LCLs incubated with or without EtxB for up to 10 hours. Equal number of cells was then harvested at different points during the time course.

Cell lysates for use in minigels were prepared by solubilising whole cell pellets in pre-mixed sample buffer (Novex) with addition of protease inhibitor (1:7 final volume) (Boehringer Mannheim) to prevent non-specific protein degradation. This was followed by boiling for 5 min and sonication for 30 sec. Lysates containing equal cell numbers (1.3×10⁵ cells/lane) were separated by electrophoresis in a 4-12% Bis-Tris gradient gel. Transfer onto nitrocellulose membrane was then performed. Blocking of non-specific sites was done using 5% non-fat milk in TBS containing 0.05% Tween. The blot was first incubated with CS1-4 (1:50) followed by HRP-conjugated goat anti-mouse IgG antibody. Finally, it was placed in a HRP substrate mixture (Pierce) and exposed to X-ray film (Kodak).

For standard SDS-polyacrylamide gels, samples were prepared in similar fashion except for a change in sample buffer (0.05M Tris-HC1 pH 6.8, 2% 2-ME, 10% glycerol, 0.01% bromophenol blue). Again, equal cell numbers (1×10⁶ cells/lane) were loaded in each lane and resolved using a 12-20% SDS-polyacrylamide gradient gel. Transfer onto nitrocellulose membrane, blocking of non-specific site and binding of CS1-4 to LMP1 was done as for the minigel system. An alkaline phosphatase-conjugated secondary antibody (Sigma) was used and detection of LMP1-specific bands was carried out by treatment with ALP substrate (Vector).

### CD8+ Lines and Target Cells

WT poly T is a polyclonal CTL line that is specific for LMP2. DA c64 is a LMP2-specific T cell clone against the target HLA-A2.01-restricted epitope CLG, which is a 9-mer peptide with the sequence CLGGLLTMV at positions 426-434 on LMP2. Autologous LCLs were used as target cells during the cytotoxic assay. These were kindly provided by Prof. A. B. Rickinson, CRC Institute for Cancer Research, University of Birmingham.

### Cytotoxic Assay

This experiment was carried in collaboration with CRC Institute of Cancer Studies, University of Birmingham. WT poly T and DA c64 were used as effectors in a standard 5-hour chromium release assay. Different groups of target cells were used after extensive washings following one of these treatments:—(1) 2-hour exposure to EtxB alone, (2) 2-hour exposure to CLG (0.1 μg/ml), washing and 2-hour incubation with EtxB or (3) overnight infection with recombinant vaccina virus encoding LMP2, washing and then a 2-hour exposure to EtxB. Control targets were prepared but without the addition of EtxB. After being prepared as stated, target cells were labelled with ⁵¹Cr for 1 hour at 37° C., washed thrice and placed together with effectors at known effort:target ratios in duplicate wells of 96-U bottom well plates. Maximum release was obtained by incubating target cells in 1% Triton-X solution instead of effector cells while treating target cells in RPMI medium alone provided spontaneous release. After 5 hours of incubation, quantification of radioactivity in the supernatant was done using a gamma counter.

### Determination of the Relative Quantities of LMP1 on LCLs by Flow Cytometry

Flow cytometry was used to determine the variability of the amount of LMP1 found on the various lymphoblastoid cell lines used. This may affect the choice of LCLs to be used in subsequent experiments.

Six LCLs, namely BER, HOMII, 110W10, IR, JG and WT18 were used in this experiment while EB4 served as the negative control. Fixation and permeabilisation of the cells prior to binding of LMP1 by CS1-4 was necessary as the antibody-binding sites are on the cytosolic C-terminal of the protein.

FIG. 1 showed that there was no significant difference between the relative amounts of LMPI among the six LCLs seen. This indicated that (1) the quantity of LMP1 needed by stable EBV-transformed B-cell populations was not dependent on the host cell and (2) the choice of LCLs used would have minimal effect on the results of subsequent experiments investigating the effects of EtxB on the antigen processing pathway of LMP1 or 2.

### Titration of EtxB by Flow Cytometry

Although the viability of LCLs has been shown previously by our lab (data not shown) to be unaffected by incubation with EtxB for up to 72 hrs, we needed to determine the optimal concentration of EtxB to be used in order to achieve maximal binding to GM1 on the cell surface.

LCLs were incubated at 1×10⁶ cells/ml with EtxB at different concentrations and analysis using flow cytometry to determine the amount of EtxB found in the LCLs was then carried out (FIG. 2).

From the results, the binding of EtxB to LCLs appeared to be maximal between the concentrations of 1.0 to 20.0 μg/ml (table 1), though the differences between these groups were small. Based on these results, EtxB was used at a concentration of 10 μg/ml for subsequent experiments.

TABLE 1
LCLs were treated with varying concentrations of EtxB as
shown. These were then labelled for EtxB and analysed
using FACS. Treatment of cells with 1.0 to 20.0 μg/ml of
EtxB produced the strongest signals, indicating higher
binding affinity of EtxB at these concentrations.

| | | | | | | |
|---|---|---|---|---|---|---|
| Concentration of EtxB | 0.0 | 0.1 | 1.0 | 10.0 | 20.0 | 30.0 |
| (μg/ml) | | | | | | |
| Geometric mean | 18.15 | 24.16 | 61.23 | 45.61 | 53.03 | 28.01 |

### Co-Capping of EtxB and LMP1 Seen by Immunofluorescence Confocal Microscopy

As previously mentioned, both GM1 and LMP1 are found on glycosphingolipid-rich (GSL) domains on cell surfaces. It was thought that capping and internalisation of EtxB following binding onto GM1 might alter the distribution of LMP1 in LCLs.

By labelling EtxB with a fluorochrome-conjugated antibody, its binding to GM1 with subsequent capping and internalisation could be demonstrated using immunofluorescence microscopy. Secondly, the colocalisation of LMP1 with EtxB as well as its redistribution following capping could also be shown by labelling LMP1 with a different fluorochrome. Confocal microscopy was used as it provided the necessary sensitivity to visualise the distribution of labelled proteins both on the cell surface and within the cell.

When cells treated at 4° C. were visualised, Texas red-labelled EtxB was found to be distributed in a punctate fashion over the cell surface (FIG. 3), indicating that binding to GM1 had occurred but no capping was seen. FITC-labelled LMP1 was also distributed predominantly over the cell surface in patches as predicted. More importantly, colocalisation of EtxB and LMP1 was seen on the cell surface when simultaneous detection of EtxB and LMP1 was performed.

At 37° C., capping of EtxB towards one pole of the cell was noted and this induced LMP1 to do likewise, i.e. co-capping had occurred (FIG. 4). It was, however, not possible to show the distribution of EtxB and LMP1 within the cell. This could be due to (1) minute quantities of EtxB and LMP1 being internalised, (2) rapid degradation of EtxB and LMP1 within the cell following internalisation, (3) difficulty in obtaining sufficiently thin ‘optical slices’ (minimum 1 μm) in relatively small LCLs (average size 10-20 μ) and (4) lack of a counter-stain to provide better demarcation between cytosolic and nuclear compartments within the cell. It is likely that postulate (1) is correct.

### No Degradation of LMP1 Seen on Western Blot Following EtxB Treatment

Based on the evidence from confocal microscopy that LMP1 colocalises with EtxB and undergoes co-capping, further investigations were necessary to show whether LMP1 does indeed become internalised with EtxB. It was thought that following internalisation, LMP1 was shunted into a novel degradation pathway and hence produce new degradation products. Removal of LMP1 or other transmembrane proteins in general is thought to require specific proteolytic enzymes creating nicks at the cytoplasmic reverse-loops separating the transmembrane domains. If this occurs, then a decrease in full-length LMP1 may be detected following resolution of total cell lysate in SDS-PAGE gel electrophoresis. Indeed, the early fragments produced in this process will still contain an intact carboxyl-terminus which is recognised by CS1-4 anti-LMP1 antibody. To investigate this gradient gels were used to improve the resolution of the bands, especially the smaller fragments of LMP1 following degradation. During the preparation of the cell lysates, care was taken to minimise the effects of endogenous proteases released during lysis by keeping the samples on ice and adding protease-inhibitors to the lysates. Equal loading of lysates was checked using Ponceau S staining of the immunoblots following transfer. In some experiments, an internal control was provided by detection of an unrelated EBV protein, EBNA in the western blots.

FIG. 5 shows a typical result obtained using 4-12% minigel. Full-length LMP1 was clearly only detected in lanes containing lysates derived from LCL while absent in the EBV-negative control cell line, EB4. There was, however, no noticeable decrease in the full-length LMP1 band despite increasing treatment times with EtxB. Also, no LMP1 fragments were detected.

Although the minigel provided good quality blots, it was limited by the amount of protein that can be loaded in each lane. Hence, a switch to standard gels was made, primarily to improve sensitivity by increasing the amount of lysate added per lane. Similarly, gradient gels (12-20%) were used for improved resolutions of the bands.

Once again, the full-length LMP1 was readily detected (FIG. 6) but there was no significant decrease in intact LMP1 or any degradation bands seen with prolonged EtxB incubation. There was an added problem of increased background in this set of immunoblots, making it difficult to make out any small fragments that may have been produced.

So far, the process of internalisation and degradation of LMP1 after EtxB has not been successfully demonstrated using confocal microscopy and western blotting. This may be due to the following reasons, (1) the hypothesis is wrong and the process of internalisation and degradation never take place, (2) the methods used so far are not of adequate sensitivity to detect the very low quantities of LMP1 being internalised and degraded, (3) the C-terminal undergoes proteolysis soon after EtxB treatment with loss of antibody-binding sites for the anti-LMP1 antibody (CS1-4) used or (4) the rapid turnover of LMP1 prevents small changes in total LMP1 within the cells to be detected using the current protocol.

### Enhanced Killing by LMP2-Specific CTL EtxB-Treated Target Cells Detected by ⁵¹Cr-Release Assay

Cytotoxic T-cells (CTL) are able to recognise antigens presented on cell surfaces in association with HLA class I molecules, even if these antigens are present in trace quantities. If it is true that addition of EtxB induces LMP1 and 2 to undergo an alternative antigen processing pathway resulting in previously protected epitopes being more efficiently presented, then cytotoxic assays using CTL lines against LMP1 or 2 acting upon EtxB-treated target cells would provide an extremely sensitive and specific measure of the effects of EtxB on the processing and presentation of these latent membrane proteins.

Several HLA class I-restricted LMP2 epitopes have been identified and CD8+ lines against LMP2 and its epitopes have been generated (Rickinson). Conversely, only one LMP1 epitope has been described to date (Khanna). For this reason, chromium release assays using LMP2 specific T-cell lines were used against EtxB-treated autologous target cells.

Two CTL lines, WT poly T and DA c64, were used in these experiments. Autologous LCLs were used as target cells were subdivided into 3 groups:—(1) treated with EtxB alone, (2) pulsed with CLG peptides prior to incubating with EtxB and (3) transfected with vaccinia virus carrying the LMP2A gene before addition of EbcB.

From table 2, we see that the addition of EtxB alone to LCL resulted in a four-fold increase in the CTL response of DA c64 clone, with a smaller but still significant two-fold increase in the polyclonal line WT poly T. Consistent with this finding was the improved activity against EtxB-treated targets that have previously been transfected with vaccinia virus carrying LMP2 (vacLMP2) compared to targets treated with vacLMP2 alone. The enhancement of LMP2-specific CTL responses by EtxB are represented more clearly in FIGS. 7 and 8.

However, treating peptide-pulsed target cells with EtxB did not enhance cytotoxic activity. One likely explanation would be that the amount of peptides used to pulse the target cells has saturated the available binding sites on the cell surface, resulting in optimal recognition and hence a maximal CTL response.

This set of results is the first piece of evidence that the treatment of EBV-infected cells with EtxB can lead to enhanced processing and presentation of LMP2, producing a marked increase in CTL responses against this protein.

TABLE 2
% of specific lysis of different targets cells using WT poly T and
DA c64. There increased activity of DA c64 against both LCL and vacLMP2-
transfected LCL that have been treated with EtxB. These results were
similarly seen with WT poly T, although to a lesser extent. LCLs that have
been previously pulsed with CLG peptide were not made more susceptible to
CTL killing despite EtxB treatment.

| | | LCL+ | | | | | | |
|---|---|---|---|---|---|---|---|---|
| Target cells | | | | CLG | CLG + | | | VacLMP2 + |
| spent/max | | Cont. | EtxB | peptide | EtxB | vacTK- | vacLMP2 | EtxB |
| Effector cells | E:T | 133/976 | 76/1112 | 65/816 | 88/1071 | 115/985 | 143/1585 | 131/1241 |
| WT poly T | 10:01 | 11 | 27 | 78 | 72 | 13 | 49 | 65 |
| WT poly T | 5:01 | 7 | 35 | 61 | 58 | 9 | 51 | 59 |
| DA c64 | 5:01 | 13 | 52 | 74 | 78 | 8 | 50 | 85 |
| DA c64 | 2:01 | 15 | 53 | 68 | 71 | 8 | 57 | 72 |

While EtxB has been used extensively to enhance the intracellular delivery of exogenous antigens either as an adjuvant in vaccine development or more recently as a fusion protein, these series of experiments have shown for the first time that it has a similar effect on endogenous proteins present on the cellular membrane.

Forming the cornerstone of the hypothesis is that EtxB would act only on membrane proteins found within the same domains as its receptor GM1. This strict criterion is met by EBV latent membrane proteins found on EBV-transformed lymphoblastoid cell lines.

It has been shown that EtxB causes a change in the distribution of LMP1 on the cell surface. More significantly, as shown in the case of LMP2, it appears to alter the antigen processing pathway, leading to a dramatic enhancement of CTL response.

Although these results are still preliminary, it nevertheless shows that EtxB is usable in the enhancement of CTL recognition and killing of targets expressing LMP2 and, possibly LMP1. Hence, this can play an important role in the immunotherapy of EBV-related tumours exhibiting a latency II pattern, in particular nasopharyngeal carcinoma and Hodgkin's disease.

### B

### EtxB and its Mutants

The non-toxic recombinant EtxB has been previously described. Two mutant forms of EtxB, namely G33D and H57A, were also used in these experiments. G33D contains a Gly-33 to Asp substitution which prevents it from binding to GM1 while H57A binds to GM1 but lacks immunomodulatory activity due to a His to Ala substitution at position 57. EtxB and the mutants described above were kindly provided by Professor T. R. Hirst at the University of Bristol.

### W6/32 and DA6.231 Supernatant

W6.32 culture supernatant was collected and used as a source of pan-HLA class I blocking antibodies while that obtained from DA6.231 was similarly used as a source of anti-HLA class II blocking antibodies.

### Peptides

Peptides corresponding to known epitopes in EBV latent antigens were produced by standard 9-fluorenylmethyoxycarbonyl (FMOC) chemistry (University of Bristol) and dissolved in DMSO at known concentrations.

### Polyclonal Cytotoxic CD8+ T-Cell Lines

Peripheral blood mononuclear cells (PBMCs) were obtained from healthy seropositive donors. CD8+ cells were positively selected using magnetic cell sorting (MACS, Miltenyi Biotec) and set aside. The rest of the PBMCs were then pulsed with 50 uM of a known peptide for 1 hour at 37° C. These were then washed thrice and pooled together with the CD8+ cells. The pooled cells were then seeded at 1×10⁶ cells/ml in complete RPMI medium supplemented with 25 ng/ml IL-7. 10 U/ml IL-2 was added on day 3. Thereafter, the cultures were fed twice weekly with growth medium containing 25 ng/ml IL-7 and 10 U/mI IL-2. On day 12, CD8+ cells were again magnetically selected and counted. The remaining cells in the culture were then pulsed with 50-100 uM of peptide for 1 hour and subsequently treated with 50 ug/ml mitomycin-C for another hour at 37° C. These were then washed thrice and added to the CD8+ cells at a responder:stimulator ratio of 2:1. These cultures were subsequently tested in cytotoxicity assays and restimulated weekly with mitomycin-C treated LCLs.

### Cytotoxicity Assays

Autologous LCLs, from the same donor as the PBMCs above, were used as target cells. These were either (1) used alone or (2) treated with 10 ug/ml EtxB or one of its mutants for 4 hours or overnight. Targets were then labelled with 70-100 uCi of radioactive chromium for 90 min. Specific peptide at a final concentration of 5 uM or DMSO solvent was added for the final 60 min. in some targets. These were then washed and incubated with effectors for 4 hours in a standard chromium release assay. For experiments involving the inhibition of HLA class I antigen presentation, the labelled targets were incubated with 1:10 of W6/32 or DA6.231 supernatant for 1 hour at room temperature before the addition of the effector cells.

### Experiment B1

### ExtB Treatment of LCLs Leads to Enhanced Recognition and Killing by LMP1 and LMP2-Specific CTLs but not by EBNA3A-Specific CTLs

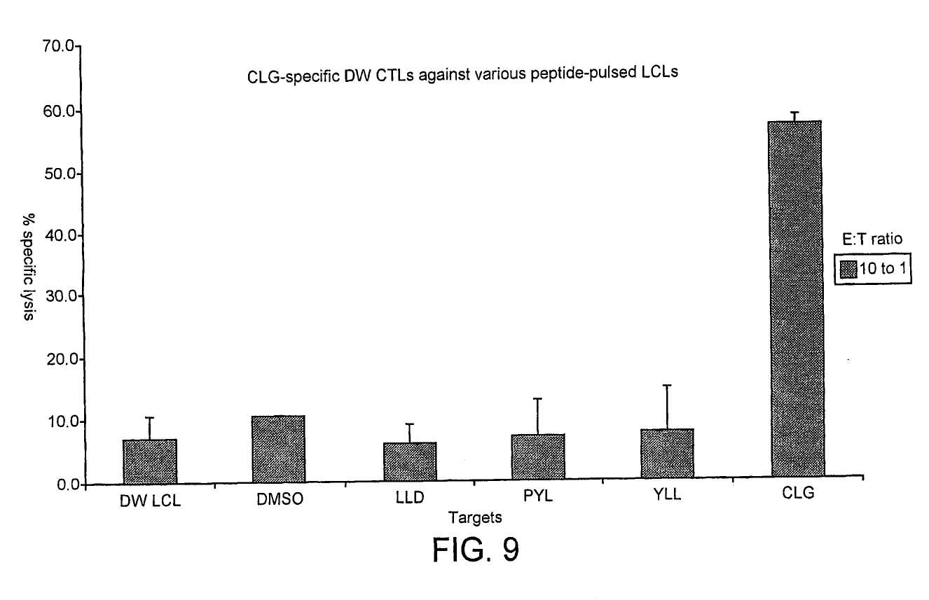

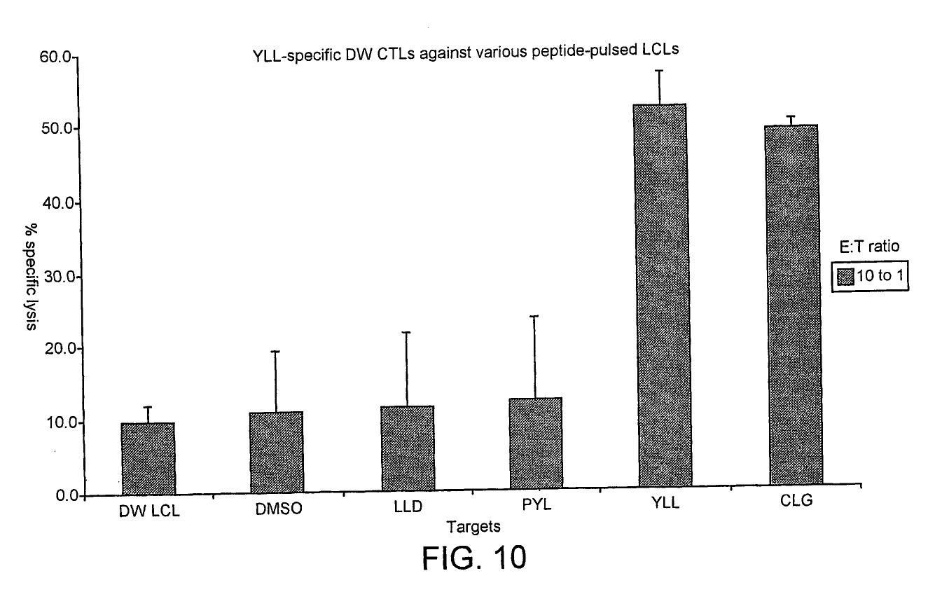

Two peptide-specific polyclonal CTL lines were raised from the donor DW (HLA-A0201, A23, B7, B49, C7). One was raised against the HLA-A2 restricted LMP1 epitope YLLEMLWRL (YLL) while the other was specific for the LMP2 epitope CLGGLLTMV (CLG) which is HLA-A2.01 restricted. These lines were tested against a range of peptides and found to be specific for the peptide they were raised against (FIGS. 9 and 10).

FIGS. 9 and 10. CLG-specific and YLL-specific DW CTL lines shown in FIGS. 9 and 10 respectively were used against autologous LCL targets used alone or pulsed with various known class I peptides or an equivalent volume of DMSO solvent. LLD represents a EBNA3C epitope, LLDFVRFMGV, which is restricted through HLA-A0201 while PYL corresponds to a HLA-A2301-restricted LMP2 epitope, PYLFWLAAI. It is clearly demonstrated that both the CTL lines tested here are specific for the peptide against which they were raised, i.e. YLL, which corresponds to a known HLA-A2 LMP1 restricted peptide YLLEMLWRL and CLG, which bears the amino-acid sequence CLGGLLTMV, which is a known HLA-A0201 restricted LMP2 epitope.

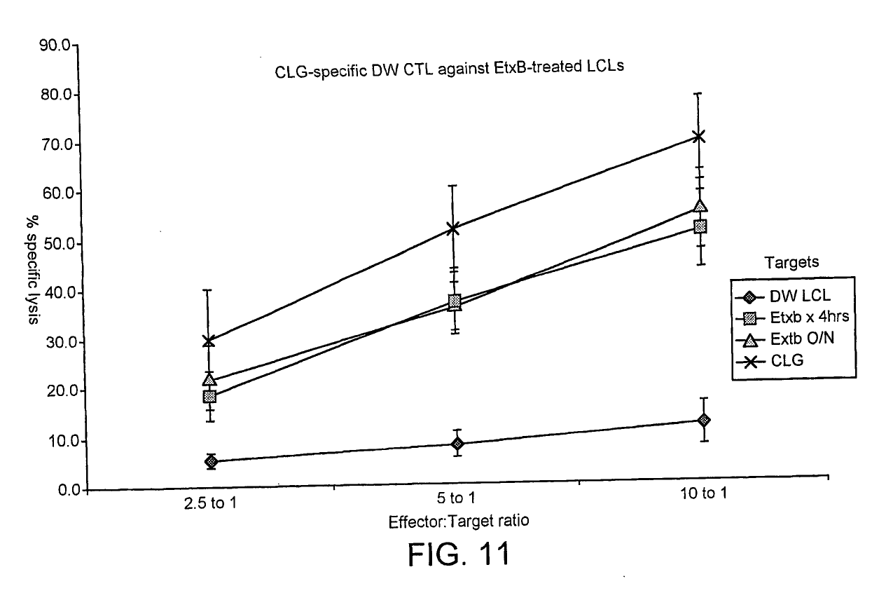

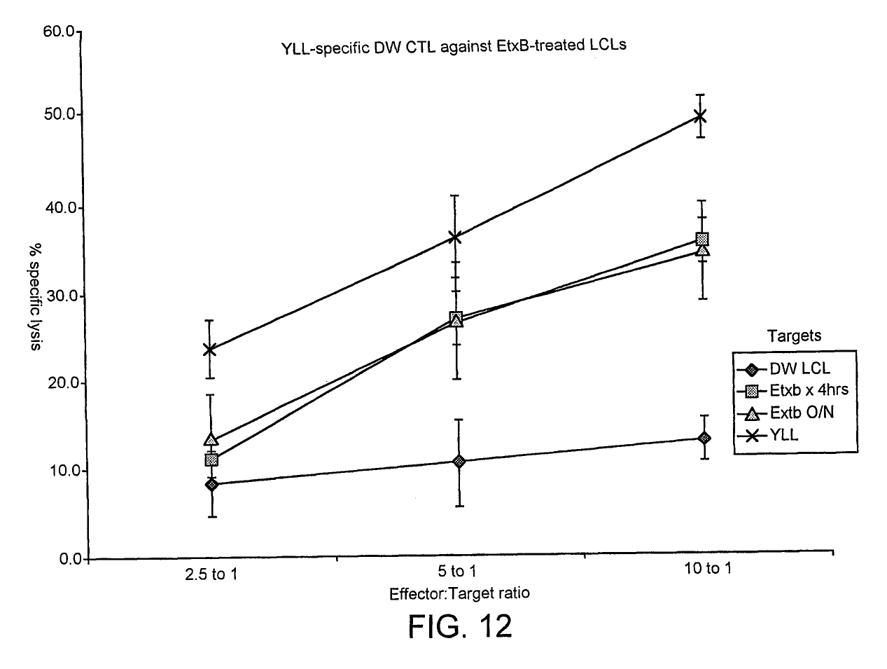

Both these lines were able to recognise and lyse autologous LCL targets which have been treated with EtxB (FIGS. 11 and 12), with the levels of killing in both lines being significantly higher than the background killing of untreated LCLs. The effects of EtxB were rapid in onset and sustained, as the enhancement in CTL recognition and killing of target cells was clearly evident after 4 hours, and was maintained at a similar level even after an overnight incubation.

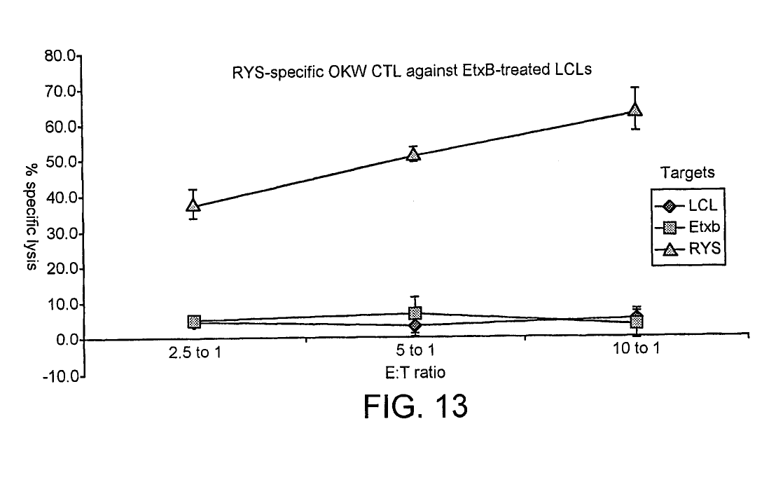

EBNA3A is another EBV latent antigen which is expressed only in the nucleus and not on the cellular membrane. A CTL line was raised from another donor OKW (HLA-A11, A24, B40, C4, C7) which was specific for RYSIFFDY (RYS), a HLA-A24 restricted EBNA3A epitope. In contrast to the CTL lines specific for LMP1 and LMP2, there is no increased killing of target LCLs treated with EtxB by the EBNA3A-specific CTL line (FIG. 13). This supports the hypothesis that EtxB is only able to modulate the antigen processing and presentation pathway of membrane bound proteins found within lipid rafts.

FIGS. 11, 12 and 13. In FIG. 11, the level of killing of EtxB-treated LCL targets by CLG-specific DW CTLs was dramatically higher than that seen against untreated LCLs. This was not due to an increase in cellular permeability as a result of EtxB treatment as the absolute counts and ratios between the spontaneous and maximal counts were largely comparable in all target groups (data not shown). This effect was similarly seen when the YLL-specific DW CTL line was used (FIG. 12). In both cases, this enhancement in cytotoxic killing was similar whether the target cells were treated with EtxB for 4 hours or after an overnight incubation. This suggests that the effects of EtxB on LMP1 and LMP2 were rapid and sustained. A control CTL line specific for RYSIFFDY (RYS), a HLA-24 restricted EBNA3A epitope was also used in similar experiments. However, there was no enhancement of cytotoxic killing despite treating target LCLs with EtxB (FIG. 13). This suggests that EtxB is only able to influence the antigen processing and presentation pathway of membrane bound antigens found.

### Experiment B2

### The Enhancement in CTL Killing of EtxB-Treated LCLs is Mediated Through HLA Class I

In order to determine if the enhancement in CTL killing following EtxB treatment is mediated through HLA class I, EtxB-treated LCLs were incubated with W6/32 supematant which contains pan HLA class I antibodies. This prevents the interaction between the T-cell receptors (TCR) on CD8+ CTLs and HLA class I molecules on the LCL targets.

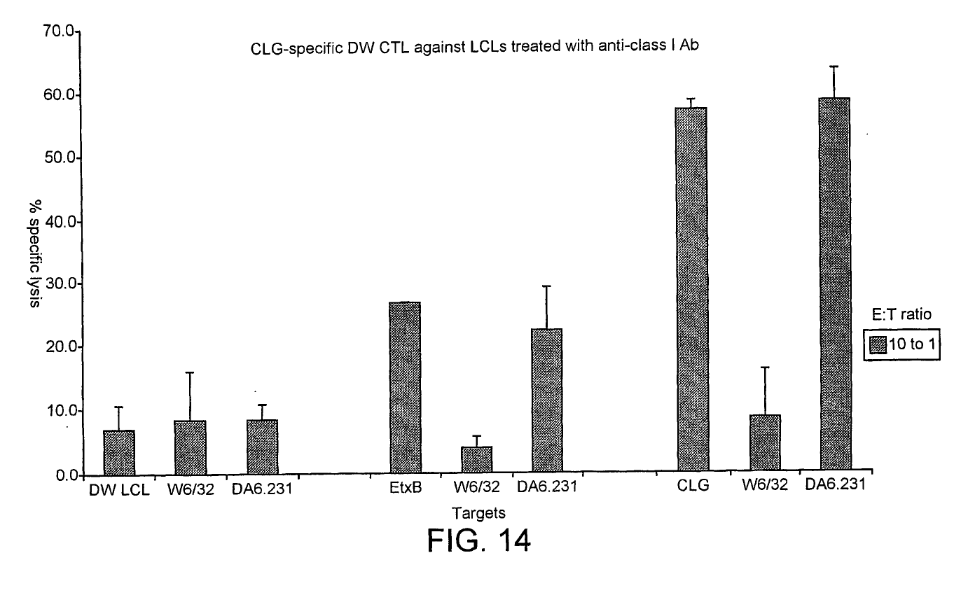

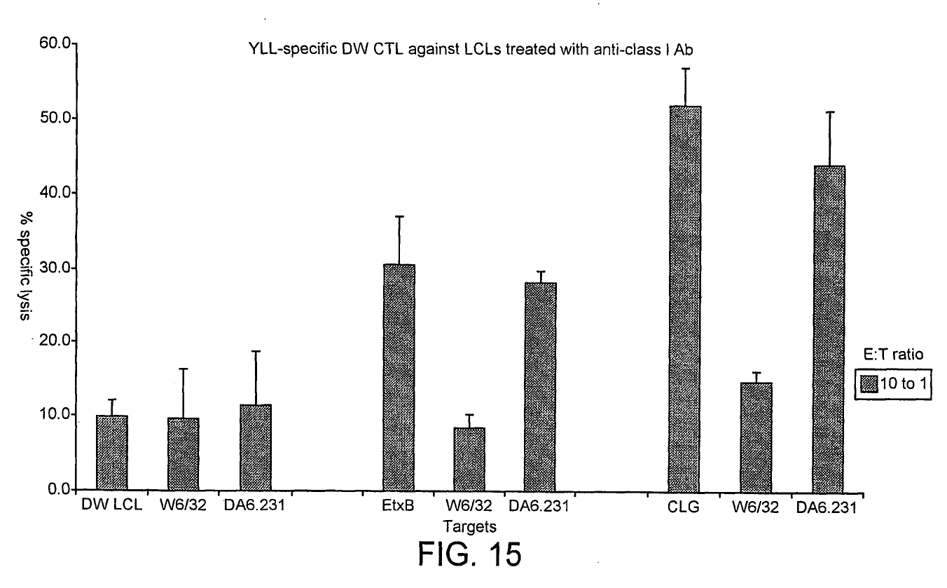

FIGS. 14 and 15. A pan-HLA class I blocking antibody, W6/32, was used following EtxB treatment or peptide-pulsing of target LCLs. The lysis of peptide-pulsed targets by the relevant CTL line was effectively blocked while those treated with an anti-HLA class II antibody, DA6.231, remained unaffected. This effect was similarly seen in the EtxB-treated group of targets, suggesting that the effects of EtxB on LMP1 and LMP2 is mediated through the HLA class I antigen presentation pathway.

As seen in FIGS. 14 and 15, following incubation with W6/32 antibodies, there was a dramatic decrease in the levels of killing by both YLL and CLG-specific DW CTLs against EtxB treated targets. A similar and expected decrease was also seen in the targets pulsed with the relevant peptides. There was no appreciable change in the effects of EtxB when DA6.231 anti-class II antibodies were used.

Hence, the enhancement in CTL killing following EtxB treatment appears to be due to the increased production of known class I epitopes in both LMP1 and LMP2 which are subsequently complexed with HLA class I molecules and presented on the surfaces of LCL targets.

### Experiment B3

### The Influence of ETxB on LMP1 and LMP2 Requires GM1 Binding but does not Depend on its Immunomodulatory Activity

EtxB is known to possess several immunomodulatory effects, such as the up-regulation of MHC class II and CD25 in B cells and the induction of apoptosis in murine CD8+ T cells through an NFkB-dependent and caspase-3-dependent pathway.

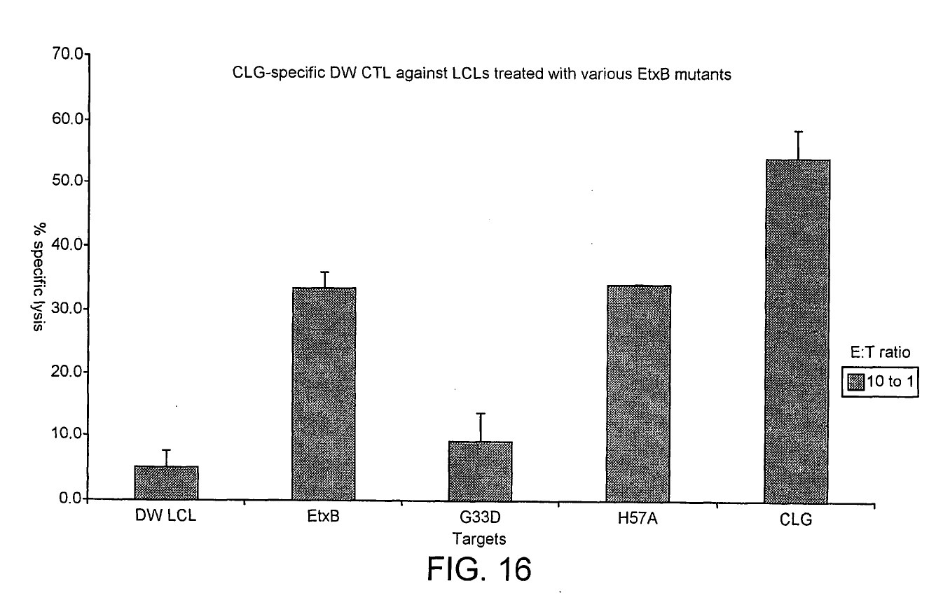

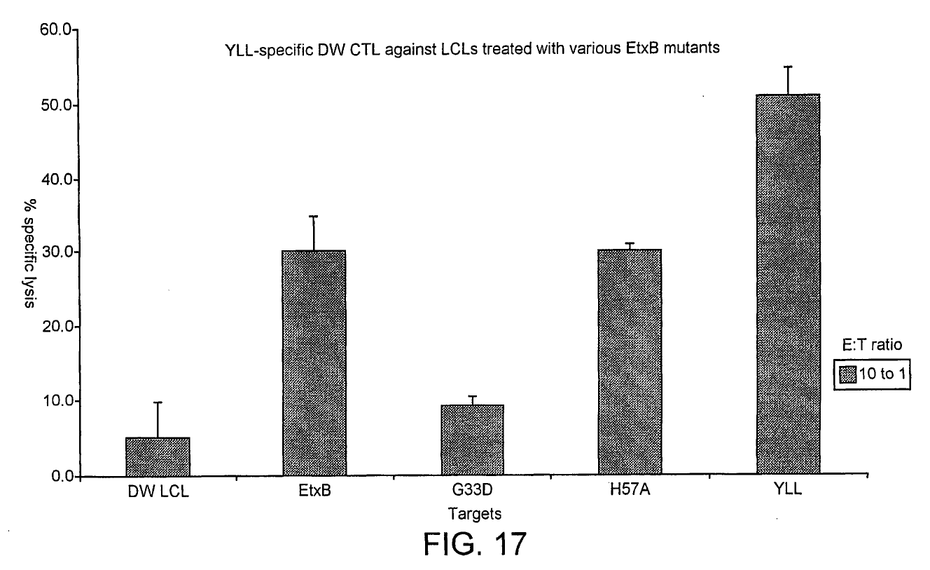

FIGS. 16 and 17. Target cells treated with G33D, a non-binding mutant of EtxB, failed to be recognised and killed by the CTL lines. This indicates that the binding of EtxB to its ganglioside receptor GM1 is required for its effects on LMP1 and LMP2. A second mutant, H57A, which binds to GM1 but does not possess known immunomodulatory effects such as induction of apoptosis in murine CD8+ T-cells which is NFkB and caspase-dependent, was also used. Here, the enhancement in CTL killing against LMP1 and LMP2 was not inhibited, suggesting that EtxB-mediated signalling does not play a role here.

From FIGS. 16 and 17, it is clear that the binding of EtxB to GM1 is crucial to its effects on LMP1 and LMP2 as there is no enhancement in CTL killing following treatment with the non-binding mutant G33D. However, the mutant H57A, that binds GM1 but lacks immunomodulatory activity, results in the enhancement of CTL killing as seen with EtxB. Hence, it appears that the effect of EtxB on the processing of LMP1 and LMP2 and subsequent production of immunogenic peptides does not depend on its immunomodulatory activity.

### REFERENCES

1. A. B. Rickinson, D. J. Moss. 1997. Human cytotoxic T lymphocyte responses to Epstein-Barr virus infection. Annu. Rev. Immunol. 15:405-31.

2. R. Khanna, S. R. Burrows, J. Nicholls, L. M. Poulsen. 1998.

Identification of cytotoxic T cell epitopes within Epstein-Barr virus (EBV) oncogene latent membrane protein 1 (LMP1): evidence for HLA A2 supertype-restricted immune recognition of EBV-infected cells by LMP1-specific cytotoxic T lymphocytes. Eur. J. Immunol. 28:451-458.

3. J. Levitskaya, M. Coram, V. Levitsky, S. Imreh, P. M. Steigerwald-Mullen, G. Klein, M. G. Kurilla, M. G. Masucci. 1995. Inhibition of antigen processing by the internal repeat region of the Epstein-Barr virus nuclear antigen 1. Nature. 375:685-688.

4. N. A. Williams, T. R. Hirst, T. O. Nashar. 1999. Immune modulation by the cholera-like enterotoxins: from adjuvant to therapeutic. Immunol. Today. 20:95-101.

5. L. Brooks, Q. Y. Yao, A. B. Rickinson, L. S. Young. 1992. Epstein-Barr virus latent gene transcription in nasopharyngeal carcinoma cells: coexpression of EBNA1, LMP1 and LMP2 transcripts. J. Virol. 66:2689-2697.

6. B. Clausse, K. Fizazi. V. Walczak, C. Tetaud, J. Wiels, T. Tursz, P.

Busson. 1997. High concentration of the EBV latent membrane protein 1 in glycosphingolipid-rich complexes from both epithelial and lymphoid cells. Virology. 228:285-293.

7. R. Longn cker, E. Kieff. 1990. A second Epstein-Barr virus membrane protein (LMP2) is expressed in latent infection and colocalizes with LMP1. J. Virol. 64:2319-2326.

8. Loregian. E. Papini, B. Satin, H. S. Marsden, T. R. Hirst, G. Palu. 1999. Intranuclear delivery of an antiviral peptide mediated by the B subunit of Escherichia coli heat-labile enterotoxin. Proc. Natl. Acad. Sci. 96:5221-5226.

1. A therapeutic agent comprising a B-subunit of a protein toxin selected from the B-subunit of E coli heat-labile enterotoxin (EtxB) and the B-subunit of Vibrio cholerae toxin (CtxB) and a cell surface-expressed viral antigen.

2. The therapeutic agent according to claim 1, wherein the B-subunit of the protein toxin and the cell surface-expressed viral antigen are linked.

3. The therapeutic agent according to claim 1, wherein the B-subunit of the protein toxin and the cell surface-expressed viral antigen are conjugated.

4. The therapeutic agent according to claim 1, comprising a fusion protein of the B-subunit of the protein toxin and the cell surface-expressed viral antigen.

5. The therapeutic agent according to any one of claims 1 to 4, wherein the cell surface-expressed viral antigen is an Epstein Barr Virus latent membrane protein.

6. The therapeutic agent according to claim 5, wherein the cell surface-expressed viral antigen is Epstein Barr Virus LMP1.

7. The therapeutic agent according to claim 5, wherein the cell surface-expressed viral antigen is Epstein Barr Virus LMP2.

8. The therapeutic agent according to any one of claims 1 to 7, wherein the B-subunit of the protein toxin is EtxB.

9. A fusion protein comprising a B-subunit of a protein toxin selected from the B-subunit of E. coli heat-labile enterotoxin (EtxB) and the B-subunit of Vibrio cholerae toxin (CtxB) and a cell surface-expressed viral antigen.

10. The fusion protein according to claim 9, wherein the cell surface-expressed viral antigen is an Epstein Barr Virus latent membrane protein.

11. The fusion protein according to claim 10, wherein the cell surface-expressed viral antigen is Epstein Barr-Virus LMP1.

12. The fusion protein according to claim 10, wherein the cell surface-expressed viral antigen is Epstein Barr Virus LMP2.

13. The fusion protein according to any one of claims 9 to 12, wherein the B-subunit of the protein toxin is EtxB.

14. A fusion protein comprising a first protein homologous to the B-subunit of either E. coli heat-labile enterotoxin (EtxB) or Vibrio cholerae toxin (CtxB) and a second protein homologous to a cell surface-expressed viral antigen, said first homologous protein being capable of binding to the GM1-receptor and said second homologous protein being capable of being internalised into a cell and altering the antigen processing pathway therein.

15. The fusion protein according to claim 14, wherein the second homologous protein is a protein homologous to Epstein Barr Virus LMP1.

16. The fusion protein according to claim 14, wherein the second homologous protein is a protein homologous to Epstein Barr Virus LMP2.

17. The use of a composition comprising a B-subunit of a protein toxin selected from the B-subunit of E. coli heat-labile enterotoxin (EtxB) and the B-subunit of Vibrio cholerae toxin (CtxB) and an Epstein Barr Virus latent membrane protein in the manufacture of a medicament for the treatment of a disease associated with the Epstein Barr Virus in an animal body, including a human body.

18. The use according to claim 17, wherein the Epstein Barr Virus latent membrane protein is LMP1.

19. The use according to claim 17, wherein the Epstein Barr Virus latent membrane protein is LMP2.

20. The use of a composition comprising a B-subunit of a protein toxin selected from the B-subunit of E. coli heat-labile enterotoxin (EtxB) and the B-subunit of Vibrio cholerae toxin (CtxB) and an Epstein Barr Virus latent membrane protein in the manufacture of a medicament for the treatment of neoplasia in an animal body, including a human body.

21. The use according to claim 20, wherein the Epstein Barr Virus latent membrane protein is LMP1.

22. The use according to claim 20, wherein the Epstein Barr Virus latent membrane protein is LMP2.

23. A method of treating an animal body, including a human body, suffering from an Epstein Barr related illness which comprises administering to the animal body, including the human body, an effective amount of the therapeutic agent clamed in any one of claims 5 to 7.

24. A method of treating an animal body, including a human body, suffering from a neoplasia which comprises administering to the animal body, including the human body, an effective amount of the therapeutic agent claimed in any one of claims 5 to 7.

25. The use of a B-subunit of a protein toxin selected from the B-subunit of E. coil heat-labile enterotoxin (EtxB) and the B sub-unit of Vibrio cholerae toxin (CtxB) to alter antigenic processing and presentation of viral and tumour antigens.

26. The use of a B-subunit of a protein toxin selected from the B-subunit of E. coli heat-labile enterotoxin (EtxB) and the B-subunit of Vibrio cholerae toxin (CtxB) in the manufacture of a medicament for the treatment of a disease or condition associated with Epstein Barr Virus in an animal body, including a human body, infected with Epstein Barr Virus.

27. The use of a B-subunit of a protein toxin selected from the B-subunit of E. coli heat-labile enterotoxin (EtxB) and the B-subunit of Vibrio cholerae toxin (CtxB) in the manufacture of a medicament for the treatment of neoplasia in an animal body, including a human body.

28. A method of treating an animal body, including a human body, suffering from an Epstein Barr Virus related illness which comprises administering to the animal body, including the human body, an effective amount of a B-subunit of a protein toxin selected from the B-subunit of E. coli heat-labile enterotoxin (EtxB) and the B-subunit of Vibrio cholerae toxin (CtxB).

29. A method of treating an animal body, including a human body, suffering from a neoplasia which comprises administering to the animal body, including the human body, an effective amount of a B-subunit of a protein toxin selected from the B-subunit of E. coli heat-labile enterotoxin (EtxB) and the B-subunit of Vibrio cholerae toxin (CtxB).
